(19) United States
(12) Patent Application Publication
(10) Pub. No.: US 2025/0215417 A1
(43) Pub. Date: Jul. 3, 2025

(54) Methods for Separating Certain Oligonucleotide Compounds

(71) Applicant: Ionis Pharmaceuticals, Inc., Carlsbad, CA (US)

(72) Inventors: Stilianos G. Roussis, Vista, CA (US); Claus Andre Frank Rentel, San Diego, CA (US)

(73) Assignee: Ionis Pharmaceuticals, Inc., Carlsbad, CA (US)

(21) Appl. No.: 18/850,238

(22) PCT Filed: Mar. 30, 2023
(86) PCT No.: PCT/US23/65130

§ 371 Date: Sep. 24, 2024

Related U.S. Application Data
(60) Provisional application No. 63/325,407, filed on Mar. 30, 2022.

(51) Int. Cl. C12N 15/10; C07H 21/04

(52) U.S. Cl. CPC C12N 15/101; C07H 21/04

(57) ABSTRACT

Provided are methods for reducing the amount of a contaminant in a sample containing an oligomeric compound. The method may comprise chromatography using a mobile phase comprising a strong salt and a chaotrope. Also provided are oligomeric compounds prepared by the method.

### FIELD

The present disclosure provides methods for separating oligomeric compounds from a mixture.

### BACKGROUND

Antisense technology is an effective means for modulating the expression of one or more specific gene products and can therefore prove to be uniquely useful in a number of therapeutic, diagnostic, and research applications. Chemically modified nucleosides may provide improvement of one or more properties, such as nuclease resistance, pharmacokinetics, or affinity for a target nucleic acid. Conjugate groups may be appended to a modified oligonucleotide to improve uptake into cells and/or tissues of interest.

Oligomeric compounds comprising an oligonucleotide and at least one conjugate group are chemically synthesized in a multi-step process that has the potential to introduce a number of unwanted contaminants. The contaminants may be other oligomeric compounds differing by minor structural modifications. The separation of desired oligomeric compounds from such contaminants remains an important challenge in bringing oligonucleotide-based therapeutics to patients.

### SUMMARY

The present disclosure provides an improved method of separating an oligomeric compound from a contaminant in a mixture, wherein the method comprises chromatography using a mobile phase comprising a strong salt and a chaotrope. In certain embodiments, the mixture of oligomeric compounds comprises a first oligomeric compound and a second oligomeric compound, wherein the second oligomeric compound is selected from a deamination product, a de-phosphorothioated product, a de-guanylated product, and a de-adenylated product of the first oligomeric compound. In certain embodiments, the method separates the first oligomeric compound from the second oligomeric compound. Also provided are oligomeric compounds prepared by the method.

### DETAILED DESCRIPTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “HPLC stationary phase” means a stationary phase used in high performance liquid chromatography. In certain embodiments, HPLC stationary phase is contained within an HPLC column. In certain embodiments, HPLC stationary phase is commercially available in pre-packed HPLC columns. In certain embodiments, HPLC stationary phase is composed of polystyrene. In certain embodiments, HPLC stationary phase is monodisperse polystyrene. In certain embodiments, HPLC stationary phase is surface-modified. In certain embodiments, HPLC stationary phase is surface-modified silica, methyl acrylate, or polystyrene. In certain embodiments, HPLC stationary phase is C18 surface-modified silica.

As used herein, “mobile phase” means a solution that flows over a stationary phase in a column. In certain embodiments, “mobile phase” is loading solution. In certain embodiments, “mobile phase” is clution solution.

As used herein, “carbohydrate” means a monosaccharide, disaccharide, trisaccharide, tetrasaccharide, oligosaccharide, polysaccharide, or derivatives thereof. In certain embodiments, a carbohydrate is N-acetylgalactosamine.

As used herein, “GalNAc” means an N-acetyl galactosamine moiety, represented by the structure:

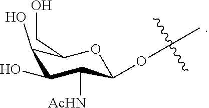

As used herein, a “deamination nucleoside” is a nucleoside containing a nucleobase:

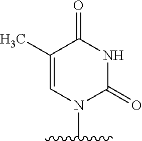 [SMILES: *n1cc(C)c(=O)[nH]c1=O]

and results from —NH₂ hydrolysis of a 5-methylcytosine. A deamination nucleoside may include a modified sugar moiety as defined herein. A “deamination product” is an oligonucleotide that contains a deamination nucleoside.

As used herein, a “de-phosphorothioated internucleoside linkage” is a phosphodiester intemucleoside linkage, and results from oxidative cleavage of a phosphorothioate intemucleoside linkage.

As used herein, a “de-guanylated nucleoside” is a 1′-hydroxyl nucleoside that results from cleavage of a guanine nucleobase.

As used herein, a “de-adenylated nucleoside” is a 1′-hydroxyl nucleoside that results from cleavage of an adenine nucleobase.

As used herein, “1′-hydroxyl sugar moiety” means a nucleoside including a sugar moiety comprising 1′—(H)(OH). The sugar moiety may be further modified, e.g., may comprise a bridge (i.e., may be a bicyclic sugar moiety) or a 2′-substitution as described herein. A “1′-hydroxyl nucleoside” means a nucleoside comprising a 1′-hydroxyl sugar moiety.

As used herein, “2′-Deoxynucleoside” means a nucleoside according to the structure:

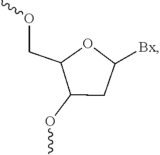

wherein Bx is a nucleobase.

As used herein, “2′-deoxy sugar moiety” means the sugar moiety of a 2′-deoxynucleoside. As indicated in the above structure, a 2′-deoxy sugar moiety can have any stereochemistry. For example, 2′-deoxy sugar moieties include, but are not limited to 2′-β-D-deoxyribosyl sugar moieties and 2′-β-D-deoxyxylosyl sugar moieties.

As used herein, “2′-β-D-deoxyribosyl nucleoside” means a nucleoside according to the structure:

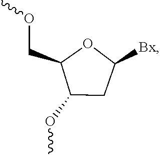

wherein Bx is a nucleobase.

As used herein, “2′-β-D-deoxyribosyl sugar moiety” means the sugar moiety of a 2′-β-D-deoxyribosyl nucleoside. The nucleobase of a 2′-deoxynucleoside or 2′-β-D-deoxyribosyl nucleoside may be a modified nucleobase or any natural nucleobase, including but not limited to an RNA nucleobase (uracil).

As used herein, “ribo-2′-MOE nucleoside” means a nucleoside according to the structure:

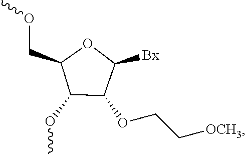

wherein Bx is a nucleobase.

As used herein, “ribo-2′-MOE sugar moiety” means the sugar moiety of a 2′-MOE nucleoside as defined herein.

As used herein, “MOE” means an —OCH₂CH₂OCH₃ group.

As used herein, “2′-OMe nucleoside” means a nucleoside according to the structure:

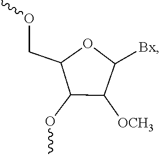

wherein Bx is a nucleobase.

As used herein, “2′-OMe sugar moiety” means the sugar moiety of a 2′-OMe nucleoside. As indicated in the above structure, a 2′-OMe sugar moiety can have any stereochemistry. For example, 2′-OMe sugar moieties include, but are not limited to 2′-OCH₃-β-D-xylosyl sugar moieties, 2′-OCH₃-α-L-ribosyl sugar moieties, and ribo-2′-OMe sugar moieties as defined herein.

As used herein, “Ribo-2′-OMe nucleoside” means a nucleoside according to the structure:

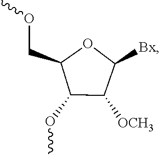

wherein Bx is a nucleobase.

As used herein, “ribo-2′-OMe sugar moiety” means the sugar moiety of a ribo-2′-OMe nucleoside.

As used herein, “2′-F nucleoside” means a nucleoside according to the structure:

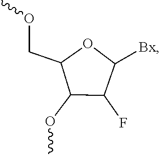

wherein Bx is a nucleobase.

As used herein, “2′-F sugar moiety” means the sugar moiety of a 2′-F nucleoside. As indicated in the above structure, a 2′-F sugar moiety can have any stereochemistry. For example, 2′-F sugar moieties include, but are not limited to, 2′-F-β-D-xylosyl sugar moieties, 2′-F-β-D-arabinosyl sugar moieties, 2′-F-α-L-ribosyl sugar moieties, and ribo-2′-F sugar moieties as defined herein.

As used herein, “ribo-2′-F nucleoside” means a nucleoside according to the structure:

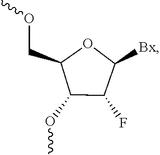

wherein Bx is a nucleobase.

As used herein, “ribo-2′-F sugar moiety” means the sugar moiety of a ribo-2′-F nucleoside as defined herein.

As used herein, “2′-NMA nucleoside” means a nucleoside according to the structure:

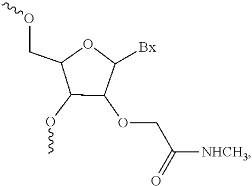

wherein Bx is a nucleobase.

As used herein, “2′-NMA sugar moiety” means the sugar moiety of a 2′-NMA nucleoside.

As used herein, “ribo-2′-NMA nucleoside” means a nucleoside according to the structure:

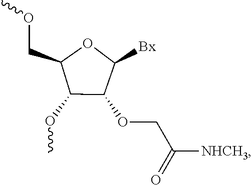

wherein Bx is a nucleobase.

As used herein, “ribo-2′-NMA sugar moiety” means the sugar moiety of a ribo-2′-NMA nucleoside.

As used herein, “2′-substituted” in reference to a sugar moiety means a furanosyl sugar moiety comprising at least one 2-substituent group other than H or OH. As used herein, “2′-substituted nucleoside” means a nucleoside comprising a 2′-substituted furanosyl sugar moiety.

As used herein, “5-methylcytosine” means a cytosine modified with a methyl group attached to the 5 position. A 5-methylcytosine is a modified nucleobase.

As used herein, “abasic sugar moiety” means a sugar moiety of a nucleoside that is not attached to a nucleobase. Such abasic sugar moieties are sometimes referred to in the art as “abasic nucleosides.”

As used herein, “bicyclic sugar” or “bicyclic sugar moiety” means a modified sugar moiety comprising two rings, wherein the second ring is formed via a bridge connecting two of the atoms in the first ring thereby forming a bicyclic structure, wherein the first ring of the bicyclic sugar moiety is a furanosyl ring. Examples of bicyclic sugar moieties include LNA (locked nucleic acid) sugar moiety and cEt sugar moiety as defined herein. A “bicyclic nucleoside” is a nucleoside comprising a bicyclic sugar moiety.

As used herein, “chaotrope” has its customary meaning in the art and means a molecule that can disrupt hydrogen bonding, hydrophobic interactions, and/or electrostatic interactions of a solute in an aqueous medium.

As used herein, “chirally enriched” in reference to a population means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom as defined herein. Chirally enriched populations of molecules having multiple chiral centers within each molecule may contain one or more stereorandom chiral centers. In certain embodiments, the molecules are modified oligonucleotides. In certain embodiments, the molecules are oligomeric compounds comprising modified oligonucleotides. In certain embodiments, the chiral center is at the phosphorous atom of a phosphorothioate internucleoside linkage. In certain embodiments, the chiral center is at the phosphorous atom of a mesyl phosphoramidate internucleoside linkage.

As used herein, “cleavable moiety” means a bond or group of atoms that is cleaved under physiological conditions, for example, inside a cell, an animal, or a human.

As used herein, “conjugate group” means a group of atoms that is directly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a covalently bound group of atoms that modifies one or more pharmacological properties of a molecule compared to the identical molecule lacking the conjugate moiety, including but not limited to pharmacodynamics, pharmacokinetics, stability, binding, absorption, tissue distribution, cellular distribution, cellular uptake, charge, and clearance.

As used herein, “constrained ethyl nucleoside” or “cEt nucleoside” means

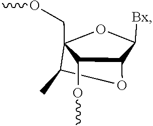

wherein Bx is a nucleobase

“Constrained ethyl” or “cEt” or “cEt sugar moiety” means the sugar moiety of a cEt nucleoside.

As used herein, “deoxy region” means a region of 5-12 contiguous nucleotides, wherein at least 70% of the nucleosides comprise a 2′-deoxy sugar moiety. In certain embodiments, a deoxy region is the gap of a gapmer.

As used herein, “internucleoside linkage” is the covalent linkage between adjacent nucleosides in an oligonucleotide. As used herein “modified internucleoside linkage” means any internucleoside linkage other than a phosphodiester internucleoside linkage.

As used herein, “linked nucleosides” are nucleosides that are connected in a contiguous sequence (i.e., no additional nucleosides are presented between those that are linked).

As used herein, “motif” means the pattern of unmodified and/or modified sugar moieties, nucleobases, and/or internucleoside linkages, in an oligonucleotide.

As used herein, “modified nucleoside” means a nucleoside comprising a modified nucleobase and/or a modified sugar moiety.

As used herein, “modified sugar moiety” means a sugar moiety of a nucleoside other than 2′-β-D-deoxyribosyl sugar moiety (the sugar moiety of unmodified DNA) or β-D-ribosyl sugar moiety (the sugar moiety of unmodified RNA).

As used herein, “non-bicyclic modified sugar moiety” means a modified sugar moiety that comprises a modification, such as a substituent, that does not form a bridge between two atoms of the sugar to form a second ring.

As used herein, “nucleobase” means an unmodified nucleobase or a modified nucleobase. A nucleobase is a heterocyclic moiety. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one other nucleobase. A “5-methylcytosine” is an example of a modified nucleobase. A universal base is a modified nucleobase that can pair with any one of the five unmodified nucleobases.

As used herein, “nucleobase sequence” means the order of contiguous nucleobases in a nucleic acid or oligonucleotide independent of any sugar or internucleoside linkage modification.

As used herein, “nucleoside” means a compound or fragment of a compound comprising a nucleobase and a sugar moiety. The nucleobase and sugar moiety are each, independently, unmodified or modified.

As used herein, “oligomeric agent” means an oligomeric compound and optionally one or more additional features, such as a second oligomeric compound. An oligomeric agent may be a single-stranded oligomeric compound or may be an oligomeric duplex formed by two complementary oligomeric compounds.

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound.

The term “oligomeric duplex” means a duplex formed by two oligomeric compounds having complementary nucleobase sequences.

As used herein, “oligonucleotide” means a strand of linked nucleosides connected via intemucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide comprising one or more modified nucleosides or having one or more modified internucleoside linkages. As used herein, “unmodified oligonucleotide” means an oligonucleotide that does not comprise any nucleoside modifications or intemucleoside modifications.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution or sterile artificial cerebrospinal fluid.

As used herein “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein, “stabilized phosphate group” means a 5′-phosphate analog that is metabolically more stable than a 5′-phosphate as naturally occurs on DNA or RNA.

As used herein, “stereorandom” or “stereorandom chiral center” in the context of a population of molecules of identical molecular formula means a chiral center that is not controlled during synthesis, or enriched following synthesis, for a particular absolute stereochemical configuration. The stereochemical configuration of a chiral center is random when it is the result of a synthetic method that is not designed to control the stereochemical configuration. For example, in a population of molecules comprising a stereorandom chiral center, the number of molecules having the (S) configuration of the stereorandom chiral center may be the same as the number of molecules having the (R) configuration of the stereorandom chiral center (“racemic”). In certain embodiments, the stereorandom chiral center is not racemic because one absolute configuration predominates following synthesis, e.g., due to the action of non-chiral reagents near the enriched stereochemistry of an adjacent sugar moiety. In certain embodiments, the stereorandom chiral center is at the phosphorous atom of a stereorandom phosphorothioate or mesyl phosphoramidate internucleoside linkage.

As used herein, “stereo-standard nucleoside” means a nucleoside comprising a non-bicyclic β-D-ribosyl sugar moiety.

As used herein, “stereo-non-standard nucleoside” means a nucleoside comprising a non-bicyclic furanosyl sugar moiety having a configuration other than that of a stereo-standard sugar moiety.

As used herein, “strong salt” means a high salt that dissociates fully in aqueous solution (having a high ionic strength).

As used herein, “sugar moiety” means any sugar moiety described herein and may be an unmodified sugar moiety or a modified sugar moiety. As used herein, “unmodified sugar moiety” means a β-D-ribosyl moiety, as found in natural RNA (an “unmodified RNA sugar moiety”), or a 2′-β-D-deoxyribosyl sugar moiety, as found in natural DNA (an “unmodified DNA sugar moiety”). As used herein, “modified sugar moiety” or “modified sugar” means a modified furanosyl sugar moiety or a sugar surrogate.

As used herein, “sugar surrogate” means a moiety that can link a nucleobase to another group, such as an internucleoside linkage, conjugate group, or terminal group in an oligonucleotide, but which is not a furanosyl sugar moiety or a bicyclic sugar moiety. Modified nucleosides comprising sugar surrogates can be incorporated into one or more positions within an oligonucleotide and such oligonucleotides are capable of hybridizing to complementary oligomeric compounds or target nucleic acids. Examples of sugar surrogates include GNA (glycol nucleic acid), FHNA (fluoro hexitol nucleic acid), morpholino, and other structures described herein and known in the art.

As used herein, “terminal group” means a chemical group or group of atoms that is covalently linked to a terminus of an oligonucleotide.

As used herein, “gapmer” means a modified oligonucleotide comprising an internal region positioned between external regions having one or more nucleosides, wherein the nucleosides comprising the internal region are chemically distinct from the nucleoside or nucleosides comprising the external regions, and wherein the modified oligonucleotide supports RNAse H cleavage. The internal region may be referred to as the “gap” and the external regions may be referred to as the “wings.” In certain embodiments, the internal region is a deoxy region. The positions of the internal region or gap refer to the order of the nucleosides of the internal region and are counted starting from the 5′-end of the internal region. Unless otherwise indicated, “gapmer” refers to a sugar motif. In certain embodiments, each nucleoside of the gap is a 2′-deoxynucleoside. In certain embodiments, the gap comprises one 2′-substituted nucleoside at position 1, 2, 3, 4, or 5 of the gap, and the remainder of the nucleosides of the gap are 2′-deoxynucleosides. As used herein, the term “MOE gapmer” indicates a gapmer having a gap comprising 2′-deoxynucleosides and wings comprising 2′-MOE nucleosides. As used herein, the term “mixed wing gapmer” indicates a gapmer having wings comprising modified nucleosides comprising at least two different sugar modifications. Unless otherwise indicated, a gapmer may comprise one or more modified intemucleoside linkages and/or modified nucleobases and such modifications do not necessarily follow the gapmer pattern of the sugar modifications.

As used herein, “cell-targeting moiety” means a conjugate group or portion of a conjugate group that is capable of binding to a particular cell type or particular cell types.

As used herein, “hybridization” means the annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

### Certain Embodiments

Embodiment 1. A method for separating a first oligomeric compound and a contaminant in a mixture, comprising:
a) providing a column comprising a stationary phase,
b) adding loading solution to the column to the column,
c) contacting the mixture with the column,
d) adding an elution solution to the column,
e) eluting the first oligomeric compound from the column, and
f) collecting an eluent fraction containing the first oligomeric compound, wherein the eluent fraction contains a reduced proportion of the contaminant relative to the mixture, compared to the first oligomeric compound;
wherein the elution solution comprises at least about 0.1 M of a strong salt, an organic solvent, and a chaotrope; and wherein the elution solution has a pH of at least about 7.

Embodiment 2. The method of embodiment 1, wherein the chaotrope is a guanidinium salt.

Embodiment 3. The method of embodiment 2, wherein the guanidinium salt is guanidinium chloride.

Embodiment 4. The method of any one of embodiments 1-3, wherein the elution solution comprises 0.05-5 M chaotrope.

Embodiment 5. The method of embodiment 4, wherein the elution solution comprises 0.1-1 M chaotrope.

Embodiment 6. The method of embodiment 4, wherein the elution solution comprises about 0.25 M chaotrope.

Embodiment 7. The method of any one of the preceding embodiments, wherein the organic solvent is methanol, acetonitrile, isopropanol, ethanol, or tetrahydrofuran.

Embodiment 8. The method of any one of the preceding embodiments, wherein the organic solvent is an alcohol.

Embodiment 9. The method of embodiment 8, wherein the alcohol is methanol.

Embodiment 10. The method of embodiment 9, wherein the elution solution comprises 50-99% methanol by volume.

Embodiment 11. The method of embodiment 10, wherein the elution solution comprises 60-85% methanol by volume.

Embodiment 12. The method of embodiment 10, wherein the elution solution comprises about 75% methanol by volume.

Embodiment 13. The method of any one of the preceding embodiments, wherein the elution solution has a pH of 8-14.

Embodiment 14. The method of any one of the preceding embodiments, wherein the elution solution has a pH of 9-13.

Embodiment 15. The method of any one of the preceding embodiments, wherein the elution solution further comprises a weak base.

Embodiment 16. The method of embodiment 15, wherein the weak base is trisodium phosphate.

Embodiment 17. The method of any one of the preceding embodiments, wherein the elution solution comprises 1-50 mM trisodium phosphate or 10-30 mM trisodium phosphate.

Embodiment 18. The method of embodiment 17, wherein the elution solution comprises about 20 mM trisodium phosphate.

Embodiment 19. The method of any one of the preceding embodiments, wherein the elution solution comprises 0.1-5 M of the strong salt.

Embodiment 20. The method of any one of the preceding embodiments, wherein the elution solution comprises 0.25-1 M of the strong salt.

Embodiment 21. The method of any one of the preceding embodiments, wherein the elution solution comprises about 0.25 M of the strong salt.

Embodiment 22. The method of any one of the preceding embodiments, wherein the strong salt is an alkali metal salt.

Embodiment 23. The method of any one of the preceding embodiments, wherein the strong salt is sodium bromide.

Embodiment 24. The method of any one of the preceding embodiments, wherein the elution solution comprises guanidinium chloride, 60-85% methanol by volume, a weak base, at least about 0.1 M of an alkali metal salt, and water.

Embodiment 25. The method of any one of the preceding embodiments, wherein the elution solution consists of 0.1-0.5 M guanidinium chloride, 60-85% methanol by volume, 10-30 mM trisodium phosphate, 0.25-1 M sodium bromide, and water.

Embodiment 26. The method of any one of the preceding embodiments, wherein the loading solution is water optionally comprising a second weak base.

Embodiment 27. The method of any one of the preceding embodiments, wherein the loading solution comprises water and 1-50 mM trisodium phosphate or 10-30 mM trisodium phosphate.

Embodiment 28. The method of embodiment 27, wherein the loading solution comprises about 20 mM trisodium phosphate.

Embodiment 29. The method of any one of the preceding embodiments, wherein at least a portion of the loading solution is added before the elution solution.

Embodiment 30. The method of any one of the preceding embodiments, wherein the loading solution and the elution solution are added together.

Embodiment 31. The method of any one of the preceding embodiments, wherein the loading solution and the elution solution are added in a gradient.

Embodiment 32. The method of any one of the preceding embodiments, wherein a ratio of loading solution to elution solution is 50 to 100% by volume.

Embodiment 33. The method of embodiment 31 or 32, wherein final proportions are 50-100%, or 60-95% by volume elution solution with the remainder being loading solution.

Embodiment 34. The method of any one of embodiments 31-33, wherein gradient begins at about 60% elution solution by volume with the remainder being loading solution, and ends at about 90% elution solution by volume, with the remainder being loading solution.

Embodiment 35. The method of any one of embodiments 31-34, wherein the gradient is a linear gradient.

Embodiment 36. The method of any one of the preceding embodiments, wherein the stationary phase comprises a resin selected from surface-modified methacrylate polystyrene, surface-modified polystyrene, surface-modified silica, and polyvinyl alcohol.

Embodiment 37. The method of embodiment 36, wherein the resin is functionalized methacrylate.

Embodiment 38. The method of embodiment 37, wherein the resin is an amine functionalized methacrylate.

Embodiment 39. The method of embodiment 38, wherein the resin is diethylaminoethyl (DEAE) functionalized methacrylate.

Embodiment 40. The method of any one of the preceding embodiments, wherein the method is conducted at 30-90° C.

Embodiment 41. The method of any one of the preceding embodiments, wherein the method is conducted at about 60° C.

Embodiment 42. The method of any one of the preceding embodiments, wherein the column is at a pressure of at least 1000 psi.

Embodiment 43. The method of any one of the preceding embodiments, wherein the first oligomeric compound comprises a modified oligonucleotide consisting of 10-30 linked nucleosides, for example 16-20 or 20 linked nucleosides, comprising adenine, cytosine, guanine, 5-methylcytosine, thymine, and/or uracil nucleobases.

Embodiment 44. The method of embodiment 43, wherein the modified oligonucleotide has a gapmer sugar motif.

Embodiment 45. The method of embodiment 43 or 44, wherein the modified oligonucleotide comprises a central region of 7-12 nucleosides flanked on the 5′-side by a 5′-external region consisting of 1-6 linked 5′-region nucleosides and on the 3′-side by a 3′-extremal region consisting of 1-6 linked 3′-region nucleosides; wherein each of the 5′-region nucleosides is a modified nucleoside, and each of the 3′-region nucleosides is a modified nucleoside.

Embodiment 46. The method of embodiment 45, wherein the central region comprises linked 2′-D-D-deoxyribosyl nucleosides, each 3′-region nucleoside is selected from a ribo-2′-MOE nucleoside, a cEt nucleoside, and a ribo-2′-OMe nucleoside, and each 5′-region nucleoside is selected from a ribo-2′-MOE nucleoside, a cEt nucleoside, and a ribo-2′-OMe nucleoside.

Embodiment 47. The method of any one of embodiments 43-46, wherein the first oligomeric compound comprises a conjugate group or a stabilized phosphate group.

Embodiment 48. The method of embodiment 47, wherein the conjugate group comprises at least one GalNAc moiety, and optionally a triantennary GalNAc cell-targeting moiety.

Embodiment 49. The method of embodiment 48, wherein the conjugate group has the structure:

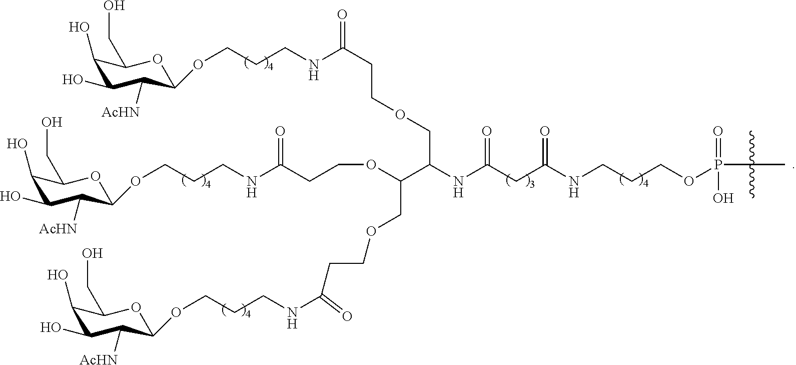

Embodiment 50. The method of any one of embodiments 1-48, wherein the first oligomeric compound consists of a modified oligonucleotide.

Embodiment 51. The method of any one of the preceding embodiments, wherein the contaminant is a second oligomeric compound.

Embodiment 52. The method of embodiment 51, wherein the second oligomeric compound comprises a deamination nucleoside, a de-phosphorothioated intemucleoside linkage, a de-guanylated nucleoside, or a de-adenylated nucleoside relative to the first oligomeric compound.

Embodiment 53. The method of embodiment 52, wherein the second oligomeric compound comprises a deamination nucleoside relative to the first oligomeric compound.

Embodiment 54. The method of embodiment 53, wherein the second oligomeric compound differs from the first oligomeric compound by only deamination nucleoside(s).

Embodiment 55. The method of embodiment 52, wherein the second oligomeric compound comprises a de-phosphorothioated internucleoside linkage relative to the first oligomeric compound.

Embodiment 56. The method of embodiment 55, wherein the second oligomeric compound differs from the first oligomeric compound by only de-phosphorothioated internucleoside linkage(s).

Embodiment 57. The method of embodiment 52, wherein the second oligomeric compound comprises a de-guanylated nucleoside relative to the first oligomeric compound.

Embodiment 58. The method of embodiment 57, wherein the second oligomeric compound differs from the first oligomeric compound by only de-guanylated nucleoside(s).

Embodiment 59. The method of embodiment 52, wherein the second oligomeric compound comprises a de-adenylated nucleoside relative to the first oligomeric compound.

Embodiment 60. The method of embodiment 59, wherein the second oligomeric compound differs from the first oligomeric compound by only de-adenylated nucleoside(s).

Embodiment 61. A method for preparing an oligomeric compound comprising a step of purifying the oligomeric compound by the method of any one of the preceding embodiments.

Embodiment 62. An oligomeric compound prepared by the method of embodiment 61.

### Certain Processes for the Purification of Oligonucleotides

The present disclosure provides high pressure chromatography conditions that achieve improved separation compared to standard purification conditions for oligomeric compounds. Specifically, the present disclosure provides an improved process of purifying oligomeric compounds from a mixture comprising at least one contaminant. The method includes eluting an oligomeric compound using an elution solution including a weak basic salt and a chaotropic agent.

Surprisingly, the instant methods successfully separate, at least partially, structurally similar oligomeric compounds. For example, the contaminant may be a second oligomeric compound arising from a side reaction in synthesis, or from a degradation process, e.g., during handling or storage. The contaminant may be an oligomeric compound having a deamination nucleoside, a de-phosphorothioated intemucleoside linkage, a de-guanylated nucleoside, or a de-adenylated nucleoside relative to the first oligomeric compound.

### Certain Purification Conditions

High-performance liquid chromatography (HPLC) is a process of separating organic molecules by flowing a sample mixture in an loading solution over a column containing a solid adsorbent material (e.g., HPLC stationary phase), followed by changing the solution flowing over the column in order to elute the material that was adsorbed onto the stationary phase. Typically, the first mobile phase is a loading solution that contains little or no organic solvent, and the mobile phase used to elute the material that is adsorbed onto the stationary phase, or elution solution, contains more organic solvent.

In certain embodiments, a gradient is used to gradually change the ratio of two or more solutions flowing over the column. For example, the gradient may be a linear gradient in which the proportions of each solution are varied at a constant rate. In certain embodiments, a step gradient is used to rapidly change the ratio of two or more solutions flowing over the stationary phase. As the amount of organic solvent increases, the adsorbed organic molecules (e.g, oligonucleotides and/or contaminants) are eluted from the column and can be collected in eluent fractions. In certain embodiments, three or more solutions are used. In certain such embodiments, a washing solution may be added to the column after the loading solution and before the elution solution. The washing solution may elute materials with retention that differs greatly from the product and close-eluting contaminants (e.g., first oligomeric compound and second oligomeric compound).

The present disclosure provides an improved process for separation of oligomeric compounds differing only by chemical change at a single nucleoside or internucleoside linkage. In some embodiments, improved chromatographic separation is observed between such oligomeric compounds when a chaotrope is included in a mobile phase such as the elution solution. In certain embodiments, the chaotrope is selected from n-butanol, ethanol, a guanidinium salt, lithium perchlorate, lithium acetate, magnesium chloride, phenol, 2-propanol, sodium dodecyl sulfate, thiourea, and urea. In certain embodiments, the chaotrope is a guanidinium salt, for example, guanidinium chloride.

In certain embodiments, the elution solution has a specific pH. The pH of the solution may be selected to increase resolution. In certain embodiments, the elution solution is pH 8-14. In certain embodiments, the elution solution is pH 9-13. In certain embodiments, the elution solution is pH 10-12, for example, about 10, about 10.5, about 11, about 11.5, or about 12. In certain embodiments, the elution solution has a pH of about 11.5. The concentration of a basic constituent in the elution solution, for example a weak basic salt, may be chosen to provide such a pH as described herein.

In certain embodiments, the elution solution comprises an organic solvent. In certain embodiments, the elution solution comprises 50-100%, or 60-95%, organic solvent by volume. In certain embodiments, the organic solvent is methanol, acetonitrile, isopropanol, ethanol, or tetrahydrofuran. In certain embodiments, the organic solvent is methanol. The elution solution may comprise, consist of, or consist essentially of guanidinium chloride, methanol, trisodium phosphate, an alkali metal salt, and water. In certain embodiments, the alkali metal salt may be sodium chloride, sodium bromide, potassium chloride, or potassium bromide. The elution solution may comprise, consist essentially of, or consist of guanidinium chloride, 60-85% methanol by volume, a weak base, at least about 0.25 M of a strong salt (e.g., an alkali metal salt), and water. The elution solution may comprise, consist of, or consist essentially of 0.1-0.5 M guanidinium chloride, 60-85% methanol by volume, 10-30 mM trisodium phosphate, 0.25-1 M sodium bromide, and water.

The column may be a typical chromatography stationary phase as known in the art, for example, an HPLC stationary phase, and may comprise a Weak Anion Exchange (WAX) chromatography stationary phase as known in the art. The WAX stationary phase is characterized by carrying a charge depending on the pH of the mobile phase in contact. The WAX stationary phase is preferably hydrophilic and may comprise a functional group capable of interacting with a proton, for example, an amine functional group. In certain embodiments, the stationary phase is selected from surface-modified methacrylate polystyrene, surface-modified polystyrene (e.g., polystyrene cross-linked with divinylbenzene), surface-modified silica, and polyvinyl alcohol. In certain embodiments, the stationary phase is a surface-modified methacrylate, for example, diethylaminoethyl (DEAE) or diethylaminopropyl (ANX) functionalized methacrylate. The stationary phase may comprise polyethyleneimine (PEI), for example, linear or branched polyethyleneimine (PEI).

The methods described herein may be implemented in analytical, preparatory, or process separations.

In certain embodiments, the methods described herein are useful for purifying mixtures containing oligomeric compounds comprising oligonucleotides consisting of linked nucleosides. Oligonucleotides may be unmodified oligonucleotides or may be modified oligonucleotides. Modified oligonucleotides comprise at least one modification relative to an unmodified oligonucleotide (i.e., comprise at least one modified nucleoside (comprising a modified sugar moiety and/or a modified nucleobase) and/or at least one modified internucleoside linkage). The present disclosure provides processes of purifying oligomeric compounds comprising oligonucleotides that have any number or combinations of modifications described herein.

Surprisingly, in certain embodiments, the combination of purification conditions described herein allow for separation of oligomeric compounds having closely-related structures differing, for example, by a single functional group, or by the presence of absence of a single nucleobase. The oligomeric compounds may be any described herein and without limitation may comprise modified oligonucleotides have lengths of 12-30, or 16-20, linked nucleosides. Such separations may be important in permitting purification and analysis of therapeutic compounds.

### I. Certain Oligonucleotides

In certain embodiments, provided herein are oligomeric compounds comprising oligonucleotides, which consist of linked nucleosides, wherein the oligomeric compound is prepared by a method described herein. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

### A. Certain Modified Nucleosides

Modified nucleosides comprise a modified sugar moiety or a modified nucleobase, or both a modified sugar moiety and a modified nucleobase.

### 1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non-bridging substituents may be at any position of the furanosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃(“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, 0-C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.).

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, and OCH₂CH₂OCH₃. In certain embodiments, a modified oligonucleotide comprises one or more of a 2′-β-D-Deoxyribosyl nucleoside, a ribo-2′-MOE nucleoside, a ribo-2′-OMe nucleoside, a ribo-2′-F nucleoside, and a ribo-2′-NMA nucleoside. In certain embodiments, the modified oligonucleotide comprises a stereo-non-standard sugar moiety.

In certain embodiments, modified furanosyl sugar moieties and nucleosides incorporating such modified furanosyl sugar moieties are further defined by isomeric configuration. For example, a 2′-deoxyfuranosyl sugar moiety may be in seven isomeric configurations other than the naturally occurring β-D-deoxyribosyl configuration. Such modified sugar moieties are described in, e.g., WO 2019/157531, incorporated by reference herein. A 2′-modified sugar moiety has an additional stereocenter at the 2′-position relative to a 2′-deoxyfuranosyl sugar moiety; therefore, such sugar moieties have a total of sixteen possible isomeric configurations. 2′-modified sugar moieties described herein are in the (3-D-ribosyl isomeric configuration unless otherwise specified.

In naturally occurring nucleic acids, sugars are linked to one another 3′ to 5′. In certain embodiments, oligonucleotides include one or more nucleoside or sugar moiety linked at an alternative position, for example at the 2′ or inverted 5′ to 3′. For example, where the linkage is at the 2′ position, the 2′-substituent groups may instead be at the 3′-position.

Certain modified sugar moieties comprise a substituent that bridges two atoms of the furanosyl ring to form a second ring, resulting in a bicyclic sugar moiety. Nucleosides comprising such bicyclic sugar moieties have been referred to as bicyclic nucleosides (BNAs), locked nucleosides, or conformationally restricted nucleotides (CRN). Certain such compounds are described in US Patent Publication No. 2013/0190383; and PCT publication WO 2013/036868. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′—(CH₂)₂-2′, 4′—(CH₂)₃-2′, 4′-CH₂—O-2′ (“LNA”), 4′-CH₂—S-2′, 4′—(CH₂)₂—O-2′ (“ENA”), 4′-CH(CH₃)—O-2′ (referred to as “constrained ethyl” or “cEt”), 4′-CH₂—O—CH₂-2′, 4′-CH₂—N(R)-2′, 4′—CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J Org. Chem., 2009, 74, 118-134), 4′-CH₂—C—(═CH₂)-2′ and analogs thereof (see e.g., Seth et al., U.S. Pat. No. 8,278,426), 4′-C(RₐR)—N(R)—O-2′, 4′—C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—; wherein: x is 0, 1, or 2; n is 1, 2, 3, or 4; each Rₐ and R_{b} is, independently selected from: H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), and sulfoxyl (S(═O)-J₁); and each J₁ and J₂ is, independently selected from: H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, and a protecting group.

Additional bicyclic sugar moieties are known in the art, see, for example: Freier et al., Nucleic Acids Research, 1997, 25(22), 4429-4443, Alback et al., J Org. Chem., 2006, 71, 7731-7740, Singh ct al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J Am. Chem. Soc., 2007, 129, 8362-8379; Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

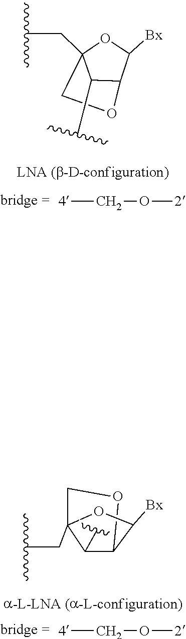

α-L-methylcncoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). The addition of locked nucleic acids to siRNAs has been shown to increase siRNA stability in serum, and to reduce off-target effects (Elmen, J. et al., (2005) Nucleic Acids Research 33(1):439-447; Mook, OR. et al., (2007) Mal Cane Ther 6(3):833-843; Grunweller, A. et al., (2003) Nucleic Acids Research 31(12):3185-3193). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“NINA”) (see, e.g., Leumann, CJ. Bioorg. & Med. Chem. 2002, 10, 841-854), fluoro HNA:

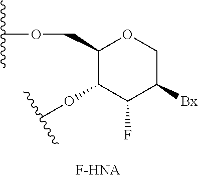

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

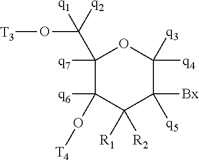

wherein, independently, for each of said modified THP nucleoside: Bx is a nucleobase moiety; T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q are each H. In certain embodiments, at least one of q₁, q₂, q₃, q₄, q₅, q₆ and q is other than H. In certain embodiments, at least one of q₁, q₂, q₃, q₄, q₅, q₆ and g₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

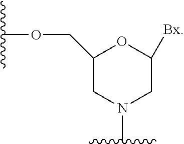

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moicites. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876. Representative U.S. patents that teach the preparation of PNA compounds include, but are not limited to, U.S. Pat. Nos. 5,539,082; 5,714,331; and 5,719,262. Additional PNA compounds suitable for use in the oligonucleotides of the invention are described in, for example, in Niclsen et al., Science, 1991, 254, 1497-1500.

In certain embodiments, sugar surrogates are the “unlocked” sugar structure of UNA (unlocked nucleic acid) nucleosides. UNA is an unlocked acyclic nucleic acid, wherein any of the bonds of the sugar has been removed, forming an unlocked sugar surrogate. Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

In certain embodiments, sugar surrogates are the glycerol as found in GNA (glycol nucleic acid) nucleosides as depicted below:

### (S)-GNA

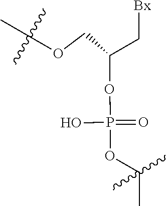

where Bx represents any nucleobase.

Many other bicyclic and tricyclic sugar and sugar surrogate ring systems are known in the art that can be used in modified nucleosides.

### 2. Certain Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside. In certain embodiments, modified oligonucleotides comprise one or more inosine nucleosides (i.e., nucleosides comprising a hypoxanthine nucleobase).

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903: Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

### 3. Certain Modified Internucleoside Linkages

The naturally occurring internucleoside linkage of RNA and DNA is a 3′ to 5′ phosphodiester linkage. In certain embodiments, nucleosides of modified oligonucleotides may be linked together using any intemucleoside linkage. The two main classes of intemucleoside linking groups are defined by the presence or absence of a phosphorus atom. Representative phosphorus-containing internucleoside linkages include but are not limited to phosphates, which contain a phosphodiester bond (“—O—P(═O)(OH)”) (also referred to as unmodified or naturally occurring linkages), phosphotriesters, methylphosphonates, phosphoramidates, and phosphorothioates (“—O—P(═S)(OH)—”), and phosphorodithioates (“—O—P(═S)(SH)”). Representative non-phosphorus containing intemucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified intemucleoside linkages, compared to naturally occurring phosphate linkages, can be used to alter, typically increase, nuclease resistance of the oligonucleotide. In certain embodiments, intemucleoside linkages having a chiral atom can be prepared as a racemic mixture, or as separate enantiomers. Methods of preparation of phosphorous-containing and non-phosphorous-containing internucleoside linkages are well known to those skilled in the art.

In certain embodiments, a modified internucleoside linkage is any of those described in WO2021/030778, incorporated by reference herein. In certain embodiments, a modified internucleoside linkage comprises the formula:

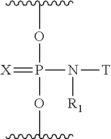

wherein independently for each internucleoside linking group of the modified oligonucleotide:
   X is selected from O or S;
   R₁ is selected from H, C₁-C₆ alkyl, and substituted C₁-C₆ alkyl; and
   T is selected from SO₂R₂, C(═O)R₃, and P(═O)R₄R₅, wherein:
   R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₂-C₆ alkenyl, C₂-C₆ alkynyl, substituted C₁-C₆ alkyl, substituted C₂-C₆ alkenyl substituted C₂-C₆ alkynyl, and a conjugate group;
   R₃ is selected from an aryl, a substituted aryl, CH₃, N(CH₃)₂, OCH₃ and a conjugate group;
   R₄ is selected from OCH₃, OH, C₁-C₆ alkyl, substituted C₁-C₆ alkyl and a conjugate group; and
   R₅ is selected from OCH₃, OH, C₁-C₆ alkyl, and substituted C₁-C₆ alkyl.

In certain embodiments, a modified internucleoside linkage comprises a mesyl phosphoramidate linking group having a formula:

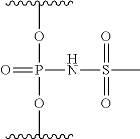 [SMILES: *OP(=O)(NS(C)(=O)=O)O*]

In certain embodiments, a mesyl phosphoramidate internucleoside linkage may comprise a chiral center. In certain embodiments, modified oligonucleotides comprising (Rp) and/or (Sp) mesyl phosphoramidates comprise one or more of the following formulas, respectively, wherein “B” indicates a nucleobase:

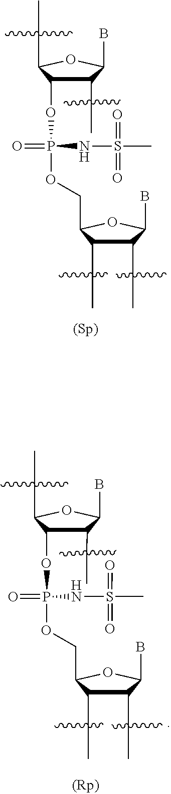

Representative internucleoside linkages having a chiral center include but are not limited to alkylphosphonates, mesyl phosphoramidates, and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate or other linkages containing chiral centers in particular stereochemical configurations. In certain embodiments, populations of modified oligonucleotides comprise phosphorothioate internucleoside linkages wherein all of the phosphorothioate internucleoside linkages are stereorandom. In certain embodiments, populations of modified oligonucleotides comprise mesyl phosphoramidate internucleoside linkages wherein all of the mesyl phosphoramidate internucleoside linkages are stereorandom. Such modified oligonucleotides can be generated using synthetic methods that result in random selection of the stereochemical configuration of each phosphorothioate linkage or mesyl phosphoramidate. Nonetheless, as is well understood by those of skill in the art, each individual phosphorothioate or mesyl phosphoramidate of each individual oligonucleotide molecule has a defined stereoconfiguration. In certain embodiments, populations of modified oligonucleotides are enriched for modified oligonucleotides comprising one or more particular phosphorothioate or mesyl phosphoramidate internucleoside linkages in a particular, independently selected stereochemical configuration. In certain embodiments, the particular configuration of the particular phosphorothioate or mesyl phosphoramidate linkage is present in at least 65% of the molecules in the population. In certain embodiments, the particular configuration of the particular phosphorothioate or mesyl phosphoramidate linkage is present in at least 70% of the molecules in the population. In certain embodiments, the particular configuration of the particular phosphorothioate or mesyl phosphoramidate linkage is present in at least 80% of the molecules in the population. In certain embodiments, the particular configuration of the particular phosphorothioate or mesyl phosphoramidate linkage is present in at least 90% of the molecules in the population. In certain embodiments, the particular configuration of the particular phosphorothioate or mesyl phosphoramidate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate or mesyl phosphoramidate in the (Sp) configuration. In certain embodiments, a population of modified oligonucleotides is enriched for modified oligonucleotides having at least one phosphorothioate or mesyl phosphoramidate in the (Rp) configuration. In certain embodiments, modified oligonucleotides comprising (Rp) and/or (Sp) phosphorothioates comprise one or more of the following formulas, respectively, wherein “B” indicates a nucleobase:

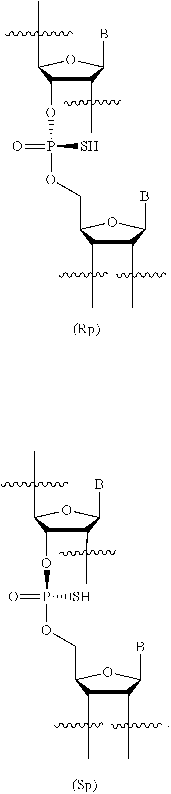

Unless otherwise indicated, chiral intemucleoside linkages of modified oligonucleotides described herein can be stereorandom or in a particular stereochemical configuration.

Neutral intemucleoside linkages include, without limitation, phosphotriesters, methylphosphonates, MMI (3′-CH₂—N(CH₃)—O-5′), amide-3 (3′-CH₂—C(═O)—N(H)—5′), amide-4 (3′-CH₂—N(H)—C(═O)—5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, modified oligonucleotides comprise one or more inverted nucleoside, as shown below:

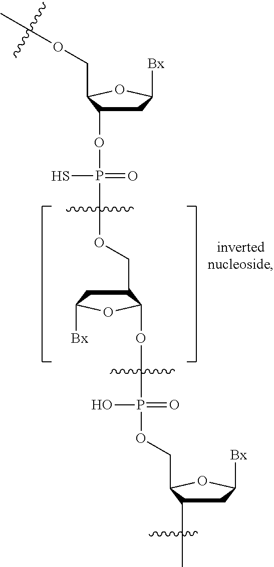

wherein each Bx independently represents any nucleobase.

In certain embodiments, an inverted nucleoside is terminal (i.e., the last nucleoside on one end of an oligonucleotide) and so only one internucleoside linkage depicted above will be present. In certain such embodiments, additional features (such as a conjugate group) may be attached to the inverted nucleoside. Such terminal inverted nucleosides can be attached to either or both ends of an oligonucleotide.

In certain embodiments, such groups lack a nucleobase and are referred to herein as inverted sugar moieties. In certain embodiments, an inverted sugar moiety is terminal (i.e., attached to the last nucleoside on one end of an oligonucleotide) and so only one internucleoside linkage above will be present. In certain such embodiments, additional features (such as a conjugate group) may be attached to the inverted sugar moiety. Such terminal inverted sugar moieties can be attached to either or both ends of an oligonucleotide.

In certain embodiments, nucleic acids can be linked 2′ to 5′ rather than the standard 3′ to 5′ linkage. Such a linkage is illustrated below.

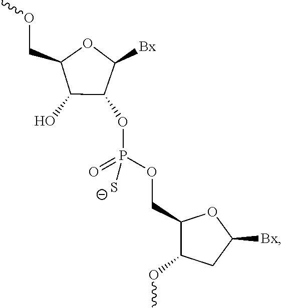

wherein each Bx represents any nucleobase.

### B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or intemucleoside linkage motif (as used herein, nucleobase motif describes the modifications to the nucleobases independent of the sequence of nucleobases).

### 1. Certain Sugar Motifs

In certain embodiments, oligomeric compounds or oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

### Uniformly Modified Oligonucleotides

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified nucleotide comprises the same 2′-modification.

### Gapmer Oligonucleotides

In certain embodiments, modified oligonucleotides comprise or consist of a sequence of nucleosides having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap region (the 3′-most nucleoside of the 5′-wing and the 5′-most nucleoside of the 3′-wing) differ from the sugar moiety of the neighboring gap nucleosides, thus defining the boundary between the wings and the gap region (i.e., the wing/gap junction). In certain embodiments, the sugar moieties within the gap are the same as one another. In certain embodiments, the gap region includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-6 nucleosides. In certain embodiments, each nucleoside of each wing region of a gapmer is a modified nucleoside. In certain embodiments, at least one nucleoside of each wing region of a gapmer is a modified nucleoside. In certain embodiments, at least two nucleosides of each wing region of a gapmer are modified nucleosides. In certain embodiments, at least three nucleosides of each wing region of a gapmer are modified nucleosides. In certain embodiments, at least four nucleosides of each wing region of a gapmer are modified nucleosides.

In certain embodiments, the gap region of a gapmer comprises 7-12 nucleosides. In certain embodiments, each nucleoside of the gap region of a gapmer is a 2′-deoxynucleoside. In certain embodiments, at least one nucleoside of the gap region of a gapmer is a modified nucleoside.

In certain embodiments, the gapmer is a deoxy gapmer, i.e., a gapmer that comprises a deoxy region. In certain embodiments, the nucleosides on the gap side of each wing/gap junction are unmodified 2′-deoxynucleosides and the nucleosides on the wing sides of each wing/gap junction are modified nucleosides. In certain embodiments, each nucleoside of the gap comprises a 2′-β-D-deoxyribosyl sugar moiety. In certain embodiments, each nucleoside of each wing of a gapmer comprises a modified sugar moiety. In certain embodiments, at least one nucleoside of the gap of a gapmer comprises a modified sugar moiety. In certain embodiments, one nucleoside of the gap comprises a modified sugar moiety and each remaining nucleoside of the gap comprises a 2′-deoxy sugar moiety. In certain embodiments, at least one, or exactly one, nucleoside of the gap of a gapmer comprises a 2′-OMe sugar moiety.

Herein, the lengths (number of nucleosides) of the three regions of a gapmer may be provided using the notation [# of nucleosides in the 5′-wing]−[# of nucleosides in the gap]−[# of nucleosides in the 3′-wing]. Thus, a 3-10-3 gapmer consists of 3 linked nucleosides in each wing and 10 linked nucleosides in the gap. Where such nomenclature is followed by a specific modification, that modification is the modification in each sugar moiety of each wing region and the gap region nucleosides comprise 2′-deoxy sugar moieties. Thus, a 3-10-3 cEt gapmer consists of 3 linked cEt nucleosides in the 5′-wing, 10 linked 2′-deoxynucleosides in the gap, and 3 linked cEt nucleosides in the 3′-wing. Similarly, a 2-12-2 cEt gapmer consists of 2 linked cEt nucleosides in the 5′-wing, 12 linked 2′-deoxynucleosides in the gap, and 2 linked cEt nucleosides in the 3′-wing. A 5-10-5 MOE gapmer consists of 5 linked ribo-2′-MOE nucleosides in the 5′-wing, 10 linked 2′-deoxynucleosides in the gap, and 5 linked ribo-2′-MOE nucleosides in the 3′-wing.

In certain embodiments, modified oligonucleotides are 5-10-5 MOE gapmers. In certain embodiments, modified oligonucleotides are 3-10-3 cEt gapmers. In certain embodiments, modified oligonucleotides are 3-10-4 MOE gapmers.

### Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methyl cytosines. In certain embodiments, all of the cytosine nucleobases are 5-methyl cytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the gap region of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynylpyrimidine.

### 2. Certain Internucleoside Linkage Motifs

In certain embodiments, oligonucleotides comprise modified and/or unmodified internucleoside linkages arranged along the oligonucleotide or region thereof in a defined pattern or motif In certain embodiments, each internucleoside linking group is a phosphodiester internucleoside linkage (“—O—P(═O)(OH)—”). In certain embodiments, each internucleoside linking group of a modified oligonucleotide is a phosphorothioate internucleoside linkage (“—O—P(═O)(SH)—”). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate a (Sp) phosphorothioate, and a (Rp) phosphorothioate.

In certain embodiments, the sugar motif of a modified oligonucleotide is a gapmer and the internucleoside linkages within the gap region are all modified. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap region comprises at least one Sp, Sp, Rp motif. In certain embodiments, populations of modified oligonucleotides are enriched for modified oligonucleotides comprising such internucleoside linkage motifs.

### C. Certain Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

### D. Certain Modified Oligonucleotides

In certain embodiments, the above modifications (sugar, nucleobase, intemucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

### E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for R-D ribosyl sugar moieties, and all of the phosphorothioate internucleoside linkages are stereorandom. In certain embodiments, the modified oligonucleotides of a chirally enriched population are enriched for both R-D ribosyl sugar moieties and at least one, particular phosphorothioate internucleoside linkage in a particular stereochemical configuration.

### F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

### II. Certain Oligomeric Compounds

In certain embodiments, provided herein is an oligomeric compound consisting of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near, e.g., one or two nucleobases from, the 3′-end of oligonucleotides. In certain embodiments, conjugate groups (or terminal groups) are attached at the 5′-end of oligonucleotides. In certain embodiments, conjugate groups are attached near, e.g., one or two nucleobases from, the 5′-end of oligonucleotides. In certain embodiments, conjugate groups (or terminal groups) are attached at the 3′-terminal nucleoside of an oligonucleotide. In certain embodiments, conjugate groups (or terminal groups) are attached at the 5′-terminal nucleoside of an oligonucleotide.

Examples of terminal groups include but are not limited to conjugate groups, capping groups, phosphate moieties, protecting groups, modified or unmodified nucleosides, and two or more nucleosides that are independently modified or unmodified.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance.

In certain embodiments, conjugation of one or more carbohydrate moieties to a modified oligonucleotide can optimize one or more properties of the modified oligonucleotide. In certain embodiments, the carbohydrate moiety is attached to a modified subunit of the modified oligonucleotide. For example, the ribose sugar of one or more ribonucleotide subunits of a modified oligonucleotide can be replaced with another moiety, e.g. a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS), which is a modified sugar moiety. A cyclic carrier may be a carbocyclic ring system, i.e., one or more ring atoms may be a heteroatom, e.g., nitrogen, oxygen, sulphur. The cyclic carrier may be a monocyclic ring system, or may contain two or more rings, e.g. fused rings. The cyclic carrier may be a fully saturated ring system, or it may contain one or more double bonds. In certain embodiments, the modified oligonucleotide is a gapmer.

In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med Chem. Lett., 1994, 4, 1053-1060), a thioether, e.g., hexyl-S-tritylthiol (Manoharan et al., Ann. N. Y. Acad. Sci., 1992, 660, 306-309; Manoharan et al., Bioorg. Med Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In certain embodiments, oligomeric compounds comprise a conjugate group comprising a cell-targeting moiety having an affinity for transferrin receptor (TfR) (also known as TfR1 or CD71). In certain embodiments, the conjugate group comprises an anti-TfR1 antibody or fragment thereof. In certain embodiments, the anti-TfR1 antibody or fragment thereof can be any known in the art including but not limited to those described in WO/1991/004753; WO/2013/103800; WO/2014/144060; WO/2016/081643; WO2016/179257; WO/2016/207240; WO/2017/221883; WO/2018/129384; WO/2018/124121; WO/2019/151539; WO/2020/132584; WO/2020/028864; U.S. Pat. Nos. 7,208,174; 9,034,329; and 10,550,188. In certain embodiments, a fragment of an anti-TfR1 antibody is F(ab′)₂, Fab, Fab′, Fv, or scFv. In certain embodiments, the conjugate group comprises a protein or peptide capable of binding TfR1. In certain embodiments, the protein or peptide capable of binding TfR1 can be any known in the art including but not limited to those described in WO/2019/140050; WO/2020/037150; WO/2020/124032; and U.S. Pat. No. 10,138,483. In certain embodiments, the conjugate group comprises an aptamer capable of binding TfR1. In certain embodiments, the aptamer capable of binding TfR1 can be any known in the art including but not limited to those described in WO/2013/163303; WO/2019/033051; and WO/2020/245198.

In certain embodiments, conjugate groups may be selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, conjugate groups may be selected from any of C22 alkyl, C20 alkyl. C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

In certain embodiments, the conjugate group has the following structure:

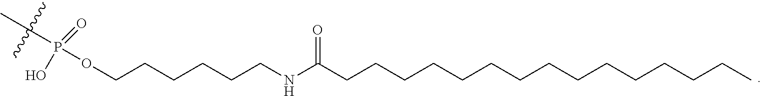 [SMILES: *P(=O)(O)OCCCCCCNC(=O)CCCCCCCCCCCCCCC]

In certain embodiments, an oligomeric compound comprises a 6-palmitamidohexyl phosphate conjugate group attached to the 5′-OH of a modified oligonucleotide wherein the structure for the conjugate group is:

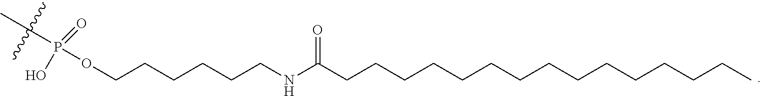 [SMILES: *P(=O)(O)OCCCCCCNC(=O)CCCCCCCCCCCCCCC]

### 1. Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen. (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### 2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises pyrrolidine.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine, or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxynucleoside that is attached to either the 3′ or 5′-terminal nucleoside of an oligonucleotide by a phosphate intemucleoside linkage and covalently attached to the remainder of the conjugate linker or conjugate moiety by a phosphate or phosphorothioate linkage. In certain such embodiments, the cleavable moiety is 2′-deoxyadenosine.

### 3. Cell-Targeting Moieties

In certain embodiments, a conjugate group comprises a cell-targeting moiety. In certain embodiments, oligomeric compounds comprise a conjugate group comprising a cell-targeting moiety having an affinity for transferrin receptor (TfR), also known as TfR1 and CD71. In certain embodiments, the conjugate group comprises an anti-TfR1 antibody or fragment thereof. In certain embodiments, the anti-TfR1 antibody or fragment thereof can be any known in the art including but not limited to those described in WO/1991/004753; WO/2013/103800; WO/2014/144060; WO/2016/081643; WO2016/179257; WO/2016/207240; WO/2017/221883; WO/2018/129384; WO/2018/124121; WO/2019/151539; WO/2020/132584; WO/2020/028864; U.S. Pat. Nos. 7,208,174; 9,034,329; and 10,550,188. In certain embodiments, a fragment of an anti-TfR1 antibody is F(ab′)₂, Fab, Fab′, Fv, or scFv.

In certain embodiments, the conjugate group comprises a protein or peptide capable of binding TfR1. In certain embodiments, the protein or peptide capable of binding TfR1 can be any known in the art including but not limited to those described in WO/2019/140050; WO/2020/037150; WO/2020/124032; and U.S. Pat. No. 10,138,483.

In certain embodiments, the conjugate group comprises an aptamer capable of binding TfR1. In certain embodiments, the aptamer capable of binding TfR1 can be any known in the art including but not limited to those described in WO/2013/163303; WO/2019/033051; and WO/2020/245198.

### B. Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

### III. Certain Oligomeric Duplexes

Certain embodiments are directed to oligomeric duplexes comprising a first oligomeric compound and a second oligomeric compound, wherein at least one of the first oligomeric compound and the second oligomeric compound is prepared by a method described herein.

Such oligomeric duplexes comprise a first oligomeric compound having a region complementary to a target nucleic acid and a second oligomeric compound having a region complementary to the first oligomeric compound. In certain embodiments, the first oligomeric compound of an oligomeric duplex comprises or consists of (1) a modified or unmodified oligonucleotide and optionally a conjugate group and (2) a second modified or unmodified oligonucleotide and optionally a conjugate group. Either or both oligomeric compounds of an oligomeric duplex may comprise a conjugate group. The oligonucleotides of each oligomeric compound of an oligomeric duplex may include non-complementary overhanging nucleosides.

In certain embodiments, an oligomeric duplex comprises: a first oligomeric compound comprising a first modified oligonucleotide a second oligomeric compound comprising a second modified oligonucleotide wherein the nucleobase sequence of the second modified oligonucleotide comprises a complementary region that is at least 90% complementary to an equal length portion of the first modified oligonucleotide.

In any of the oligomeric duplexes described herein, at least one nucleoside of the first modified oligonucleotide and/or the second modified oligonucleotide can comprise a modified sugar moiety. Examples of suitable modified sugar moieties include, but are not limited to, a bicyclic sugar moiety, such as a 2′-4′ bridge selected from —O—CH₂—; and —O—CH(CH₃)—, and a non-bicyclic sugar moiety, such as a 2′-MOE sugar moiety, a 2′-F sugar moiety, a 2′-OMe sugar moiety, or a 2′-NMA sugar moiety. In certain embodiments, at least 80%, at least 90%, or 100% of the nucleosides of the first modified oligonucleotide and/or the second modified oligonucleotide comprises a modified sugar moiety selected from 2′-F and 2′-OMe.

In any of the oligomeric duplexes described herein, at least one nucleoside of the first modified oligonucleotide and/or the second modified oligonucleotide can comprise a sugar surrogate. Examples of suitable sugar surrogates include, but are not limited to, morpholino, peptide nucleic acid (PNA), glycol nucleic acid (GNA), and unlocked nucleic acid (UNA). In certain embodiments, at least one nucleoside of the first modified oligonucleotide comprises a sugar surrogate, which can be a GNA.

In any of the oligomeric duplexes described herein, at least one internucleoside linkage of the first modified oligonucleotide and/or the second modified oligonucleotide can comprise a modified internucleoside linkage. In certain embodiments, the modified internucleoside linkage is a phosphorothioate internucleoside linkage. In certain embodiments, at least one of the first, second, or third internucleoside linkages from the 5′ end and/or the 3′ end of the first modified oligonucleotide comprises a phosphorothioate linkage. In certain embodiments, at least one of the first, second, or third internucleoside linkages from the 5′ end and/or the 3′ end of the second modified oligonucleotide comprises a phosphorothioate linkage.

In any of the oligomeric duplexes described herein, at least one internucleoside linkage of the first modified oligonucleotide and/or the second modified oligonucleotide can comprise a phosphodiester internucleoside linkage.

In any of the oligomeric duplexes described herein, each internucleoside linkage of the first modified oligonucleotide and/or the second modified oligonucleotide can be independently selected from a phosphodiester or a phosphorothioate internucleoside linkage.

In any of the oligomeric duplexes described herein, at least one nucleobase of the first modified oligonucleotide and/or the second modified oligonucleotide can be modified nucleobase. In certain embodiments, the modified nucleobase is 5-methylcytosine.

In any of the oligomeric duplexes described herein, the first modified oligonucleotide can comprise a stabilized phosphate group attached to the 5′ position of the 5′-most nucleoside. In certain embodiments, the stabilized phosphate group comprises a cyclopropyl phosphonate or an (E)-vinyl phosphonate.

In any of the oligomeric duplexes described herein, the first modified oligonucleotide can comprise a conjugate group. In certain embodiments, the conjugate group comprises a conjugate linker and a conjugate moiety. In certain embodiments, the conjugate group is attached to the first modified oligonucleotide at the 5′-end of the first modified oligonucleotide. In certain embodiments, the conjugate group is attached to the first modified oligonucleotide at the 3′-end of the modified oligonucleotide. In certain embodiments, the conjugate group comprises N-acetyl galactosamine. In certain embodiments, the conjugate group comprises a cell-targeting moiety having an affinity for transferrin receptor (TfR), also known as TfR1 and CD71. In certain embodiments, the conjugate group comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, conjugate groups may be selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, conjugate groups may be selected from any of C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

In any of the oligomeric duplexes described herein, the second modified oligonucleotide can comprise a conjugate group. In certain embodiments, the conjugate group comprises a conjugate linker and a conjugate moiety. In certain embodiments, the conjugate group is attached to the second modified oligonucleotide at the 5′-end of the second modified oligonucleotide. In certain embodiments, the conjugate group is attached to the second modified oligonucleotide at the 3′-end of the modified oligonucleotide. In certain embodiments, the conjugate group comprises N-acetyl galactosamine. In certain embodiments, the conjugate group comprises a cell-targeting moiety having an affinity for transferrin receptor (TfR), also known as TfR1 and CD71. In certain embodiments, the conjugate group comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, conjugate groups may be selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, conjugate groups may be selected from any of C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

### Nonlimiting Disclosure and Incorporation by Reference

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although sequences may be designated as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

Also encompassed by the disclosure are equivalents of the described embodiments. With regard to structure for example, stationary and mobile phases, equivalents reach those structures that allow for at least some separation of two or more oligonucleotides of typical lengths which vary only at a single sugar moiety, nucleobase, or internucleoside linkage.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Design of Model Compounds Containing Phosphorothioate Internucleoside Linkages and Associated Co-Eluting Impurities

Modified oligonucleotides having stereo-standard nucleosides were synthesized using standard techniques. Compound No. 1 is a 5-10-5 MOE gapmer containing A, ^{m}C, G, and T nucleobases, and wherein each intemucleoside linkage is a phosphorothioate internucleoside linkage. Compound No. 2 is a 5-10-5 MOE gapmer containing A, ^{m}C, G, and T nucleobases, and wherein each internucleoside linkage is a phosphorothioate internucleoside linkage. Compound No. 3 is a 3-10-4 MOE gapmer containing A, ^{m}C, G, and T nucleobases, and wherein each internucleoside linkage is a phosphorothioate internucleoside linkage. Compound No. 4 is a 5-10-5 MOE gapmer containing A, ^{m}C, G, and T nucleobases, and wherein each internucleoside linkage is a phosphorothioate internucleoside linkage. Compound No. 5 is a 5-10-5 MOE gapmer containing A, ^{m}C, G, and T nucleobases, and wherein each internucleoside linkage is a phosphorothioate internucleoside linkage, and including a LICA-1 joined by a phosphate linkage at the 5′-terminal hydroxyl group of the nucleotide as shown below:

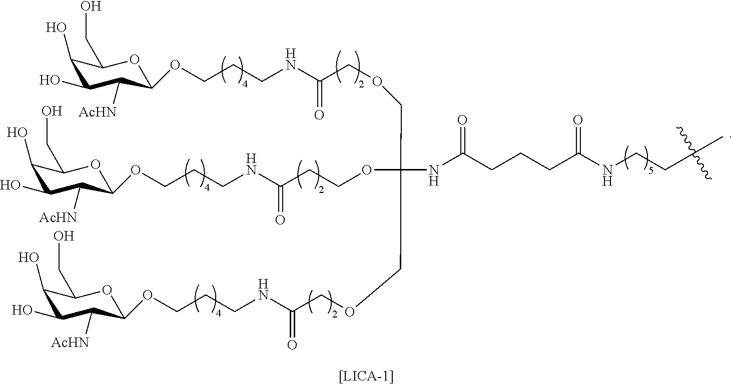

Modified oligonucleotides having stereo-standard nucleosides were designed based on parent Compound 1 and synthesized using standard techniques. Compound [1-p(dA)] is identical to Compound 1 except that a single adenine nucleoside is replaced with a 1′-hydroxyl nucleoside. Compound [1-p(dG)] is identical to Compound 1 except that one or two guanine nucleoside(s) is replaced with a 1′-hydroxyl nucleoside. Compound [1-PO] is identical to Compound 1 except that one phosphorothioate internucleoside linkage is replaced with a phosphodiester internucleoside linkage. Compound [1-DA] is identical to Compound 1 except that one to five 5-methylcytosine nucleobase(s) is replaced with a thymine nucleobase(s).

### Example 2: Development of a Method to Improve Separation of Co-Eluting Impurities in Modified Oligonucleotides Containing Phosphorothioate Internucleoside Linkages

A series of weak anion exchange (WAX) methods were tested to improve the separation of co-eluting impurities found in modified oligonucleotides containing phosphorothioate internucleoside linkages. Compound No. 1 and its impurities Compound No. [1-p(dA)], Compound No. [1-p(dG)], Compound No. [1-PO], Compound No. [1-DA](described herein above) were used as model compounds.

### Method Development

WAX HPLC analysis was performed using a Waters (Milford, MA, USA) Gen-Pak FAX (4.6×100 mm, 2.5 μm) column based on a diethylaminoethyl (DEAE) functionalized non-porous methacrylate resin. Experiments were conducted using an Agilent 1260 Infinity II liquid chromatography (LC) instrument equipped with a quaternary pump, heated column chamber, autosampler, and diode array UV detector, or an Agilent 1290 Infinity LC instrument with a binary pump, heated column chamber, autosampler, and multiple wavelength UV detector. HPLC grade acetonitrile (ACN), and ACS grade methanol (MeOH) and glacial acetic acid were acquired from J. T. Baker (Phillipsburg, NJ). Distilled, deionized water of low metal ion content (Mediatech Cellgro Sterile WFI) was purchased from VWR (Radnor, PA). All other reagents were purchased from Millipore-Sigma (St. Louis, MO).

Mobile phase A consisted of distilled, deionized water with or without 20 mM trisodium phosphate (Na₃PO₄), and 0.5 M guanidinium chloride (GnDCl). Mobile phase B (MPB) consisted of MPB stock solution combined with 0-75% of an organic solvent. The MPB stock solution consisted of 20 mM Na₃PO₄, 0.25-1.0 M NaBr, and 0.25-1.0 M GnDCl, with a pH of approximately 11.4. Samples containing model Compound No. 1 and its impurities Compound No. [1-p(dA)], Compound No. [1-p(dG)], Compound No. [1-PO], and Compound No. [1-DA](described herein above) were injected at a volume of 2 μL of 0.1 mg/mL and analyzed using the method conditions summarized in the table below.

TABLE 1
Summary of methods tested, sample volume: 2 μL of 0.1 mg/mL

| | Mobile | | Mobile Phase B | | | | | | | | |
|---|---|---|---|---|---|---|---|---|---|---|---|
| | Phase A | | MPB Stock Solution | | | | | Flow | | | |
| | Na₃PO₄ | GnDCl | Na₃PO₄ | NaBr | GnDCl | | T | Rate | Gradient | Ramp | Hold |
| Method | (mM) | (M) | (mM) | (M) | (M) | Composition | (° C.) | (mL/min) | (% B) | (min) | (min) |
| 1 | 20 mM | None | 20 | 1 | None | 100% stock | 30 | 0.3 | 40-95 | 15 | 5 |
| | Na₃PO₄ | | | | | | | | | | |
| 2 | 20 mM | 0.5 | 20 | 1 | 0.5 | 100% stock | 30 | 0.3 | 10-90 | 20 | 7 |
| | Na₃PO₄ | | | | | | | | | | |
| 3 | 0 | None | 20 | 1 | 1.0 | 100% stock | 30 | 0.6 | 10-50 | 30 | 5 |
| 4 | 0 | None | 20 | 0.7 | 0.7 | 30% ACN | 50 | 0.6 | 10-60 | 30 | 5 |
| 5 | 0 | None | 20 | 0.6 | 0.6 | 40% ACN | 60 | 0.6 | 30-60 | 30 | 5 |
| 6 | 0 | None | 20 | 0.5 | 0.5 | 50% ACN | 60 | 0.6 | 30-60 | 30 | 5 |
| 7 | 0 | None | 20 | 0.25 | 0.25 | 75% MeOH | 60 | 0.6 | 60-90 | 30 | 5 |

Retention time and peak widths of components at each of the conditions described herein above in Table 1 were calculated in OpenLab ChemStation Version C.01.09., and their resolution with respect to Compound No. 1 was calculated using the following equation in Excel: Resolution=2*(t_{R}B-t_{R}A)/(PeakWidthA+PeakWidthB), where t_{R} is retention time. The chromatographic resolutions of each impurity with respect to Compound No. 1 for each run are presented in the table below. “N. S.” indicates that no separation from Compound No. 1 was achieved. The results summarized in Table 2 indicate that Method 7, described herein above, resulted in the best overall resolution of Compound No. 1 from the impurities.

TABLE 2
Resolution of each impurity with respect to Compound
No. 1 in the method conditions described above

Resolution
(vs Compound No. 1)

| Method | [1-p(dA)] | [1-PO] | [1-DA] | [1-p(dG)] |
|---|---|---|---|---|
| 1 | N.S. | 0.67 | N.S. | 0.88 |
| 2 | 0.40 | 1.05 | 0.70 | 1.53 |
| 3 | 0.45 | 1.20 | 0.47 | 1.53 |
| 4 | 1.24 | 1.59 | 0.73 | 1.80 |
| 5 | 1.29 | 1.75 | 1.05 | 2.07 |
| 6 | 1.45 | 1.72 | 1.02 | 2.37 |
| 7 | 1.57 | 1.54 | 1.26 | 3.43 |

### Example 3: Detection of Methylated-Cytosine Deamination Using a WAX HPLC UV Method

The ability to measure deamination of methylated-cytosines in modified oligonucleotide samples using WAX HPLC UV analysis was evaluated.

### UV Signal Linearity

The linearity of the UV response relative to sample load was assessed. A sample of Compound No. 1 was spiked with 0.5-5.0% of impurity compound as indicated in the table below. The samples were analyzed by WAX HPLC using Method 7, described herein above, and a sample volume of 2 μL of 1.0 mg/mL. The impurity UV peak at 260 nm from each injection was integrated in OpenLab ChemStation version C.01.09. Blank signal was subtracted from the peak integration area, and linear regression analysis was performed in Excel. Results are presented in the table below. The results indicated that the UV response was linear over a range of sample loads from approximately 1 μg to 10 ug.

TABLE 3
UV response and linearity of impurities measured
at 260 nm, using WAX HPLC Method 7

| Compound | Impurity Peak Area | | | | |
|---|---|---|---|---|---|
| Number | 0.5% | 1.0% | 2.0% | 5.0% | R² |
| [1-PO] + [1-p(dA)] | 17.6 | 26.3 | 45.8 | 96.2 | 0.9992 |
| [1-DA] | 3.6 | 6 | 11.2 | 34.9 | 0.9984 |
| [1-p(dG)] | 3.3 | 7.5 | 13.8 | 38 | 0.9916 |

### UV Determination of Deamination in Thermal Stressed Conditions: WAX HPLC Vs HR TOF-MS

To assess the ability of the WAX HPLC method to detect deamination of methylated-cytosine residues in phosphorothioate-containing modified oligonucleotides, Compound No. 1 was subjected to thermal stress. Compound No. 1 was incubated at 80° C. for 7 days at a concentration of 200 mg/mL and pH 8. After incubation, the thermally stressed samples were analyzed by: (a) by WAX HPLC, and (b) high-resolution time-of-flight mass spectrometry (HR TOF-MS).

Experiments were conducted using an Agilent 1200 high performance liquid chromatography (HPLC) instrument equipped with a binary pump, online degasser, heated column chamber, autosampler, and multiple wavelength UV detector. The WAX HPLC experiments were conducted using conditions described herein above. For the HR TOF-MS method, the samples were analyzed by HPLC using a Waters (Milford, MA, USA) XBridge C18 (2.1×150 mm, 3.5 μm) column, temperature of 50° C., and an injection volume of 90 μL of 1 mg/mL. Mobile phase A consisted of 10% acetonitrile, 5 mM tributyl ammonium acetate, and 1 μM EDTA; and mobile phase B consisted of 80% acetonitrile, 5 mM tributyl ammonium acetate, and 1 μM EDTA. The gradient employed is described in the table below.

TABLE 4
Gradient for HR TOF-MS analysis, injection
volume 90 μL of 1 mg/mL, 50° C.

| Time (min) | Mobile Phase A (%) | Mobile Phase B (%) |
|---|---|---|
| 0.0 | 100 | 0 |
| 1.0 | 100 | 0 |
| 3.0 | 45 | 55 |
| 20.0 | 35 | 65 |
| 25.0 | 5 | 95 |
| 27.8 | 5 | 95 |
| 28.0 | 100 | 0 |
| 33.0 | 100 | 0 |

Column eluate was introduced directly into the ESI-TOF-MS. The ESI source was operated in negative mode, scanning from m/z 1000-2000 with the following instrument parameters: Capillary voltage=4000 V; drying gas temperature=320° C.; drying gas flow=12 L/min; nebulizer pressure=27 psig; fragmentor voltage=150 V; skimmer voltage=65 V; RF peak-to-peak voltage on octopole 1=250 V. The MS output signal was monitored and processed using Agilent MassHunter Qualitative Analysis software.

The area of the deamination product peak at 260 nm was integrated in OpenLab ChemStation Version C.01.09 as described above. Deamination content (DA) was calculated in Excel using the following equation: DA=(DeaminationIntegration−Blank)/(Totallntegration−Blank). Linear regression analysis was performed in Excel. The results are presented as deamination content (%) and are summarized in the table below. The WAX HPLC UV results were consistent with the values obtained using the HR TOF-MS method, indicating that deamination of methylated-cytosines in modified oligonucleotides containing phosphorothioate linkages can be quantified by the WAX HPLC UV method.

TABLE 5
Deamination of Compound No. 1 at 80° C.,
analyzed at 260 nm using WAX HPLC and HR TOF-MS

| | Deamination Content (%) | | | | | | | |
|---|---|---|---|---|---|---|---|---|
| Method | Day 0 | Day 1 | Day 3 | Day 4 | Day 7 | Slope | Intercept | R² |
| WAX HPLC | 1.28 | 3.92 | 9.80 | 11.95 | 21.40 | 2.8621 | 1.0807 | 0.9981 |
| HR TOF-MS | 0.68 | 3.28 | 8.97 | 11.24 | 20.69 | 2.8475 | 0.4304 | 0.9979 |

### Deamination Content

Modified oligonucleotides were subjected to thermal degradation followed by separation and analysis by WAX HPLC UV in order to monitor deamination in phosphorothioate-containing modified oligonucleotides.

Model compounds (described herein above) were incubated at 80° C. for an amount of time indicated in the table below. The compounds were incubated at a concentration of 200 mg/mL at pH 8. After incubation, the samples were analyzed by WAX HPLC UV using the conditions in Method 7 and a sample volume of 10 μL of 0.1 mg/mL. The area of the deamination product peak at 260 nm was integrated in OpenLab ChemStation Version C.01.09 as described above. Deamination (DA) content was calculated in Excel using the following equation: DA=(Deaminationlntegration−Blank)/(TotalIntegration−Blank). Linear regression analysis was performed in Excel. The deamination content was then divided by the number of methylated-cytosine residues to obtain the deamination per methylated-cytosine residue (%). The results are presented as deamination (%) and are summarized in the table below.

Deamination content values were predicted for given time points using Excel by using the slope and intercept values for WAX HPLC reported in Table 5. This value was divided by the methylated-cytosine content of Compound No. 1 to give the predicted deamination per methylated-cytosine, then multiplied by the number of methylated-cytosine residues in the test compound.

The WAX HPLC UV results were consistent with the predicted values, indicating that the deamination content under approximately the same thermal stressed conditions is almost constant and depends on the number of methylated-cytosine residues in the sequence. Multiple peaks were observed in compounds which were stressed for 9 days, indicating that double deamination may have occurred. This may have caused the difference between predicted and measured deamination content for Compound No. 3 and Compound No. 5.

TABLE 6
Deamination of modified oligonucleotides at 80° C., analyzed
at 260 nm using WAX HPLC Method 7, compared to predicted results

| | | | Predicted | | Measured | |
|---|---|---|---|---|---|---|
| Compound | ^{m}C | | Deamination | Deamination | Deamination | Deamination |
| Number | Residues | Days | Content (%) | per ^{m}C (%) | Content (%) | per ^{m}C (%) |
| 1 | 5 | 7 | 26.39 | 5.28 | 23.06 | 4.60 |
| 2 | 4 | 7 | 21.12 | 5.28 | 22.96 | 5.74 |
| 4 | 5 | 8 | 29.97 | 5.99 | 28.15 | 5.62 |
| 3 | 6 | 9 | 40.26 | 6.71 | 35.26 | 5.87 |
| 5 | 11 | 9 | 73.81 | 6.71 | 66.03 | 6.00 |

1. A method for separating a first oligomeric compound and a contaminant in a mixture, comprising:
a) providing a column comprising a stationary phase,
b) adding loading solution to the column to the column,
c) contacting the mixture with the column,
d) adding an elution solution to the column,
e) eluting the first oligomeric compound from the column, and
f) collecting an eluent fraction containing the first oligomeric compound,
wherein the eluent fraction contains a reduced proportion of the contaminant relative to the mixture, compared to the first oligomeric compound;
wherein the elution solution comprises at least about 0.1 M of a strong salt, an organic solvent, and a chaotrope; and wherein the elution solution has a pH of at least about 7.

2. The method of claim 1, wherein the chaotrope is a guanidinium salt.

3. The method of claim 2, wherein the guanidinium salt is guanidinium chloride.

4. The method of any one of claims 1-3, wherein the elution solution comprises 0.05-5 M chaotrope.

5. The method of claim 4, wherein the elution solution comprises 0.1-1 M chaotrope.

6. The method of claim 4, wherein the elution solution comprises about 0.25 M chaotrope.

7. The method of any one of the preceding claims, wherein the organic solvent is methanol, acetonitrile, isopropanol, ethanol, or tetrahydrofuran.

8. The method of any one of the preceding claims, wherein the organic solvent is an alcohol.

9. The method of claim 8, wherein the alcohol is methanol.

10. The method of claim 9, wherein the elution solution comprises 50-99% methanol by volume.

11. The method of claim 10, wherein the elution solution comprises 60-85% methanol by volume.

12. The method of claim 10, wherein the elution solution comprises about 75% methanol by volume.

13. The method of any one of the preceding claims, wherein the elution solution has a pH of 8-14.

14. The method of any one of the preceding claims, wherein the elution solution has a pH of 9-13.

15. The method of any one of the preceding claims, wherein the elution solution further comprises a weak base.

16. The method of claim 15, wherein the weak base is trisodium phosphate.

17. The method of any one of the preceding claims, wherein the elution solution comprises 1-50 mM trisodium phosphate or 10-30 mM trisodium phosphate.

18. The method of claim 17, wherein the elution solution comprises about 20 mM trisodium phosphate.

19. The method of any one of the preceding claims, wherein the elution solution comprises 0.1-5 M of the strong salt.

20. The method of any one of the preceding claims, wherein the elution solution comprises 0.25-1 M of the strong salt.

21. The method of any one of the preceding claims, wherein the elution solution comprises about 0.25 M of the strong salt.

22. The method of any one of the preceding claims, wherein the strong salt is an alkali metal salt.

23. The method of any one of the preceding claims, wherein the strong salt is sodium bromide.

24. The method of any one of the preceding claims, wherein the elution solution comprises guanidinium chloride, 60-85% methanol by volume, a weak base, at least about 0.1 M of an alkali metal salt, and water.

25. The method of any one of the preceding claims, wherein the elution solution consists of 0.1-0.5 M guanidinium chloride, 60-85% methanol by volume, 10-30 mM trisodium phosphate, 0.25-1 M sodium bromide, and water.

26. The method of any one of the preceding claims, wherein the loading solution is water optionally comprising a second weak base.

27. The method of any one of the preceding claims, wherein the loading solution comprises water and 1-50 mM trisodium phosphate or 10-30 mM trisodium phosphate.

28. The method of claim 27, wherein the loading solution comprises about 20 mM trisodium phosphate.

29. The method of any one of the preceding claims, wherein at least a portion of the loading solution is added before the elution solution.

30. The method of any one of the preceding claims, wherein the loading solution and the elution solution are added together.

31. The method of any one of the preceding claims, wherein the loading solution and the elution solution are added in a gradient.

32. The method of any one of the preceding claims, wherein a ratio of loading solution to elution solution is 50 to 100% by volume.

33. The method of claim 31 or 32, wherein final proportions are 50-100%, or 60-95% by volume elution solution with the remainder being loading solution.

34. The method of any one of claims 31-33, wherein gradient begins at about 60% elution solution by volume with the remainder being loading solution, and ends at about 90% elution solution by volume, with the remainder being loading solution.

35. The method of any one of claims 31-34, wherein the gradient is a linear gradient.

36. The method of any one of the preceding claims, wherein the stationary phase comprises a resin selected from surface-modified methacrylate polystyrene, surface-modified polystyrene, surface-modified silica, and polyvinyl alcohol.

37. The method of claim 36, wherein the resin is functionalized methacrylate.

38. The method of claim 37, wherein the resin is an amine functionalized methacrylate.

39. The method of claim 38, wherein the resin is diethylaminoethyl (DEAE) functionalized methacrylate.

40. The method of any one of the preceding claims, wherein the method is conducted at 30-90° C.

41. The method of any one of the preceding claims, wherein the method is conducted at about 60° C.

42. The method of any one of the preceding claims, wherein the column is at a pressure of at least 1000 psi.

43. The method of any one of the preceding claims, wherein the first oligomeric compound comprises a modified oligonucleotide consisting of 10-30 linked nucleosides, for example 16-20 or 20 linked nucleosides, comprising adenine, cytosine, guanine, 5-methylcytosine, thymine, and/or uracil nucleobases.

44. The method of claim 43, wherein the modified oligonucleotide has a gapmer sugar motif.

45. The method of claim 43 or 44, wherein the modified oligonucleotide comprises a central region of 7-12 nucleosides flanked on the 5′-side by a 5′-external region consisting of 1-6 linked 5′-region nucleosides and on the 3′-side by a 3′-extremal region consisting of 1-6 linked 3′-region nucleosides; wherein each of the 5′-region nucleosides is a modified nucleoside, and each of the 3′-region nucleosides is a modified nucleoside.

46. The method of claim 45, wherein the central region comprises linked 2′-β-D-deoxyribosyl nucleosides, each 3′-region nucleoside is selected from a ribo-2′-MOE nucleoside, a cEt nucleoside, and a ribo-2′-OMe nucleoside, and each 5′-region nucleoside is selected from a ribo-2′-MOE nucleoside, a cEt nucleoside, and a ribo-2′-OMe nucleoside.

47. The method of any one of claims 43-46, wherein the first oligomeric compound comprises a conjugate group or a stabilized phosphate group.

48. The method of claim 47, wherein the conjugate group comprises at least one GalNAc moiety, and optionally a triantennary GalNAc cell-targeting moiety.

49. The method of claim 48, wherein the conjugate group has the structure:

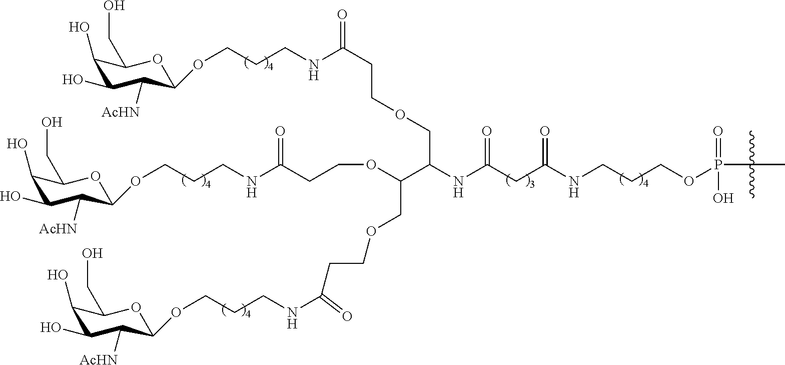

50. The method of any one of claims 1-48, wherein the first oligomeric compound consists of a modified oligonucleotide.

51. The method of any one of the preceding claims, wherein the contaminant is a second oligomeric compound.

52. The method of claim 51, wherein the second oligomeric compound comprises a deamination nucleoside, a de-phosphorothioated internucleoside linkage, a de-guanylated nucleoside, or a de-adenylated nucleoside relative to the first oligomeric compound.

53. The method of claim 52, wherein the second oligomeric compound comprises a deamination nucleoside relative to the first oligomeric compound.

54. The method of claim 53, wherein the second oligomeric compound differs from the first oligomeric compound by only deamination nucleoside(s).

55. The method of claim 52, wherein the second oligomeric compound comprises a de-phosphorothioated intemucleoside linkage relative to the first oligomeric compound.

56. The method of claim 55, wherein the second oligomeric compound differs from the first oligomeric compound by only dc-phosphorothioated intemuclcosidc linkage(s).

57. The method of claim 52, wherein the second oligomeric compound comprises a de-guanylated nucleoside relative to the first oligomeric compound.

58. The method of claim 57, wherein the second oligomeric compound differs from the first oligomeric compound by only de-guanylated nucleoside(s).

59. The method of claim 52, wherein the second oligomeric compound comprises a de-adenylated nucleoside relative to the first oligomeric compound.

60. The method of claim 59, wherein the second oligomeric compound differs from the first oligomeric compound by only de-adenylated nucleoside(s).

61. A method for preparing an oligomeric compound comprising a step of purifying the oligomeric compound by the method of any one of the preceding claims.

62. An oligomeric compound prepared by the method of claim 61.
